(19) United States
(12) Patent Application Publication
(10) Pub. No.: US 2026/0097024 A1
(43) Pub. Date: Apr. 9, 2026

(54) COMPOSITIONS FOR AND METHODS TO TREAT, PREVENT, OR REVERSE METABOLIC SYNDROME, TYPE 2 DIABETES, AND/OR CARDIOVASCULAR DISEASE

(71) Applicant: TRIMFIT FOR LIFE, LLC, Columbia, SC (US)

(72) Inventors: Roger Catarino, Columbia, SC (US); William Kuhne, Columbia, SC (US)

(21) Appl. No.: 19/352,374

(22) Filed: Oct. 7, 2025

Related U.S. Application Data
(60) Provisional application No. 63/704,177, filed on Oct. 7, 2024.

(51) Int. Cl. A61K 31/4375; A61K 31/202; A61P 3/04

(52) U.S. Cl. CPC A61K 31/4375; A61K 31/202; A61P 3/04

(57) ABSTRACT

The invention provides methods and compositions for treating, preventing, reducing the severity of, and/or reversing one or more of obesity, metabolic syndrome, and/or one or more comorbidities thereof. The compositions described herein comprise plant-based DHA and berberine in synergistically effective amounts.

### CROSS-REFERENCE TO RELATED APPLICATION

The present application relies on the disclosure of and claims priority to and the benefit of the filing date of U.S. Provisional Application No. 63/704,177, filed Oct. 7, 2024, the disclosure of which is hereby incorporated by reference herein in its entirety.

### BACKGROUND OF THE INVENTION

### Field of the Invention

The field of the invention relates to the use of a composition comprising a high specific blend of Berberine HCl (e.g., from Berberis root) and DHA (docosahexaenoic acid powder; e.g., from schizochytrium) (preferably, plant-based) for use in treating or preventing one or more disease, condition, or symptom thereof.

Specifically, the present invention is directed to compositions comprising Berberine HCl and DHA for the purpose of treating or preventing one or more of metabolic syndrome, obesity, type 2 diabetes, and/or cardiovascular disease. In embodiments, the treatment includes reduction of one or more symptom of the disease or condition, such as lowering blood sugar (e.g., allowing diabetic patients to maintain appropriate blood sugar levels without medications), building lean muscle, reducing body fat, increasing metabolism (without sugar, caffeine, or stimulants), and/or reducing cholesterol.

### Description of Related Art

Obesity, diabetes, cardiovascular disease, and accumulation of visceral fat have increased at alarming rates in the United States and abroad. The Center for Disease Control reported 71% percent of adults in the United States for the period from 2014 to 2016 were overweight or obese.

From 2020 to 2023, that number has risen to 73%. Excess body fat accumulation and obesity can result in a number of comorbid conditions, including type 2 diabetes, hypertension, and cardiovascular disease. For example, the prevalence of diabetes in the United States has risen from 6.3% in 2004 to 8.3% in 2020, as reported by the National Diabetes Statistics Report and the CDC.

With the exception of the present invention, very little success has been achieved in efforts toward lowering blood sugar, building lean muscle, lowering cholesterol, and reducing cardiovascular disease without the use of expensive pharmaceuticals.

The following patents, published patent applications, and non-patent literature are incorporated herein by reference.

Berberine is an alkaloid found in a variety of trees, herbs, and other plants, such as the barberry (berberis) shrub, golden seal, Oregon grape, tree turmeric, and Chinese goldthread. It has been reported to have anti-inflammatory effects, reduce cholesterol, and act as a preventative for several diseases and conditions, including metabolic syndrome, type 2 diabetes, obesity, cardiovascular disease, and cancer (Och, A. et al. Berberine, a Herbal Metabolite in the Metabolic Syndrome: The Risk Factors, Course, and Consequences of the Disease. Molecules. 2022 February; 27 (4) 1351).

Docosahexaenoic acid (DHA) is an omega-3 fatty acid found in the brain and retinas. DHA can be obtained through consumption of fish, fish oil, algae oil, or breastmilk or can synthesized by the body from alpha linolenic acid (ALA). It is well-known for its benefits during fetal and infant development and prevention of pre-term birth, and has additionally been shown to prevent cardiovascular disease, modulate gut microbiota, and improve cognitive and eye health (Li, J. et al. Health benefits of docosahexaenoic acid and its bioavailability: A review. Food Science & Nutrition. 2021; 9:5229-5243).

While the above compounds have been shown individually to treat or prevent one or more conditions, a need remains for highly effective compositions capable of providing faster and/or more effective treatments for obesity, metabolic syndrome, diabetes, and cardiovascular disease.

### SUMMARY OF THE INVENTION

The present invention relates to a combination of, in embodiments, all-natural, plant-based Berberine and DHA (OMEGA 3). The experimental studies conducted of each all-natural, plant-based ingredient-together and alone-showed virtually no (or minimal) negative side effects and highly superior health benefits to the consumer. In aspects, vegan capsules can be used, rather than more dangerous injections as required by prescription drugs that only accomplish a small percentage of what the product described herein offers in terms of safety, accessibility, affordability, and efficacy.

The present invention provides compositions and methods for treating or preventing type 2 diabetes and/or cardiovascular disease and/or for promoting an increase in metabolism, healthy body fat loss, increase in lean muscle mass, and/or lowering blood sugar. In a preferred embodiment, the invention is a product that can be safely, effectively, and easily administered on a daily basis.

Embodiments of the present invention include compositions and methods to provide patients with a decrease in percentage of body fat, increase in lean muscle mass, and/or other benefits of an increased or decreased metabolism.

Embodiments of the present invention include compositions and methods for increasing metabolic rate.

Embodiments of the present invention include compositions and methods for preventing, treating, and/or reversing type 2 diabetes (and/or type 1 diabetes) or the progression thereof, such as by lowering blood sugar and/or stabilizing blood sugar.

Embodiments of the present invention include compositions and methods capable of stabilizing and/or lowering blood sugar, such as by mitigating insulin resistance.

Embodiments of the present invention include compositions and methods capable of speeding wound healing.

Embodiments of the present invention include compositions and methods for preventing, treating, and/or reversing cardiovascular disease or the progression thereof.

Embodiments of the present invention include compositions comprising DHA and berberine in synergistically effective amounts for treatment, prevention, or reversal of one or more of type 2 diabetes, obesity, and/or cardiovascular disease.

Embodiments of the present invention include compositions comprising an omega-3 fatty acid, such as DHA, and berberine in synergistically effective amounts for one or more of reducing blood sugar, increasing metabolism, promoting lean muscle growth, and/or reducing body fat.

Aspect 1 is a composition comprising: a pharmaceutically acceptable salt of berberine; docosahexaenoic acid (DHA); and optionally, one or more pharmaceutically acceptable excipients.

Aspect 2 is the composition of Aspect 1, wherein the pharmaceutically acceptable salt of berberine is present in the composition in an amount in the range of about 800 mg to about 1,250 mg, and preferably around 1,000 mg.

Aspect 3 is the composition of Aspects 1 or 2, wherein the DHA is present in the composition in an amount in the range of about 150 mg to about 350 mg, and preferably around 200 mg or 250 mg.

Aspect 4 is the composition of any of Aspects 1-3, wherein the pharmaceutically acceptable salt of berberine is berberine chloride.

Aspect 5 is the composition of any of Aspects 1-4, wherein the composition is formulated as an oral dosage.

Aspect 6 is the composition of any of Aspects 1-5, wherein the oral dosage is a solid form or a liquid form.

Aspect 7 is the composition of any of Aspects 1-6, wherein the oral dosage is a pill, capsule, or tablet.

Aspect 8 is a method, comprising: administering to a subject in need thereof a composition comprising: a pharmaceutically acceptable salt of berberine; and docosahexaenoic acid (DHA); wherein the administering is performed to treat, prevent, reduce, or reverse one or more symptoms of one or more disease or condition.

Aspect 9 is the method of Aspect 8, wherein the pharmaceutically acceptable salt of berberine is present in the composition in an amount in the range of about 800 mg to about 1,250 mg, and preferably around 1,000 mg.

Aspect 10 is the method of Aspect 8 or 9, wherein the DHA is present in the composition in an amount in the range of about 150 mg to about 350 mg, and preferably around 200 mg or 250 mg.

Aspect 11 is the method of any of Aspects 8-10, wherein the pharmaceutically acceptable salt of berberine is berberine chloride.

Aspect 12 is the method of any of Aspects 8-11, wherein the composition is administered at least once per day, such as twice per day, three times per day, four times per day, or more.

Aspect 13 is the method of any of Aspects 8-12, wherein the pharmaceutically acceptable salt of berberine and the DHA are present in the pharmaceutical composition in synergistically effective amounts.

Aspect 14 is the method of any of Aspects 8-13, wherein composition is administered to the subject for a period of up to 8 weeks.

Aspect 15 is the method of any of Aspects 8-14, wherein composition is administered to the subject for a period of at least 4 weeks.

Aspect 16 is the method of any of Aspects 8-15, wherein the disease, condition, or symptom is obesity.

Aspect 17 is the method of any of Aspects 8-16, wherein the disease, condition, or symptom is metabolic syndrome.

Aspect 18 is the method of any of Aspects 8-17, wherein the disease, condition, or symptom is cardiovascular disease.

Aspect 19 is the method of any of Aspects 8-18, wherein the disease, condition, or symptom is a slowed metabolism.

Aspect 20 is the method of any of Aspects 8-19, wherein the disease, condition, or symptom is type 2 diabetes.

Aspect 21 is the method of any of Aspects 8-20, wherein the disease, condition, or symptom is elevated blood sugar.

Aspect 22 is the method of any of Aspects 8-21, wherein, as a result of the administering, the subject experiences a decrease in body fat percentage, a decrease in body mass, and/or a reduction in waist circumference.

Aspect 23 is the method of any of Aspects 8-22, wherein as a result of the administering, the subject experiences an increase in lean muscle mass.

Aspect 24 is the method of any of claims 8-23, wherein as a result of the administering, the subject experiences an increased rate of wound healing.

Aspect 25 is a method of increasing metabolic rate comprising administering to a subject the composition of any of claims 1-7.

### DETAILED DESCRIPTION

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Embodiments comprising various features may also consist of or consist essentially of those various features. Other embodiments of the invention will be apparent to those skilled in the art from consideration of the specification and practice of the invention. The description of the invention provided is merely exemplary or explanatory in nature and, thus, variations that do not depart from the essence of the invention are intended to be within the scope of the invention.

Before explaining at least one embodiment of the invention in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

The term “about” used herein in the context of quantitative measurements means the indicated amount ±10%. For example, with a ±10% range, “about 100 mg” can mean 90-110 mg.

As used herein, “treating” or “treatment” means causing a complete cure or an incomplete cure, or it means that one or more symptoms of the underlying disease or one or more comorbidities are reduced and/or that one or more of the underlying cellular, physiological, or biochemical causes or mechanisms causing the symptoms are reduced.

The term “effective amount” refers to an amount that is sufficient to affect treatment, as defined above.

Compositions disclosed may comprise one or more pharmaceutically acceptable excipient including, but not limited to, binders, fillers, diluents, aqueous solutions, organic solvents, permeation enhancers, solubilizers, disintegrants, lubricants, glidants, adjuvants, or combinations thereof. In embodiments, the composition comprises one or more of cellulose, microcrystalline cellulose, Hypromellose, silica, magnesium stearate, or combinations thereof.

Obesity, metabolic syndrome, and their associated comorbidities, such as cardiovascular disease, diabetes, high blood pressure, high cholesterol, slow metabolism, and stroke, are a significant problem worldwide. Pharmaceutical solutions have thus far proved to be dangerous, invasive, expensive, and/or result in a range of negative side effects.

Individuals categorized as overweight or obese have been shown to have higher levels of leptin, lower levels of adiponectin, slower metabolism, and/or thyroid problems. These conditions can create, cause, and/or lead to, obesity and metabolic syndrome.

The present invention provides compositions and methods of administering said compositions for the prevention, treatment, reduction, and/or reversal of one or more of obesity, metabolic syndrome, and/or one or more associated comorbidities. In embodiments, the compositions described herein are natural, mostly natural, plant-based, mostly plant-based, plant-derived, non-invasive, or mostly non-invasive, affordable, or mostly affordable, and have no, minimal, and/or no meaningful or significant, negative side effects.

In embodiments, the composition is capable of reducing the leptin level of a subject to within a normal range, such as within the range of 0.5 to 15.2 ng/ml for adult women and 0.5 to 12.5 ng/mL for adult men. In embodiments, the composition is capable of reducing leptin level by at least 5%, such as at least 10%, 15%, 20%, 25%, 30%, 35%, 40%, 50%, 60%, 70%, or more.

In embodiments, the composition is capable of increasing the subject's adiponectin level, such as to a level within the range of 2 to 37 μg/mL. In embodiments, the composition is capable of increasing a subject's adiponectin level by at least 5%, such as at least 10%, 15%, 20%, 25%, 30%, 35%, 40%, 50%, 60%, 70%, or more.

Compositions according to embodiments of the invention comprise berberine and DHA in synergistically effective amounts.

In embodiments, the composition comprises an omega-3 fatty acid chosen from DHA, alpha-linolenic acid (ALA), eicosapentaenoic acid (EPA), or combinations thereof. In embodiments, the omega-3 fatty acid(s) is obtained from a plant source, such as algae or algae oil, such as from Schizochytrium sp. In other embodiments, the omega-3 fatty acid(s) is obtained from fish oil.

In embodiments, the composition comprises berberine hydrochloride. In embodiments, the berberine hydrochloride is obtained from Berberis aristata root.

In embodiments, the composition comprises berberine, or a pharmaceutically acceptable salt thereof, in an amount of about 100 mg to 2,000 mg, such as about 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,050 mg, 1,100 mg, 1,150 mg, 1,200 mg, 1,300 mg, 1,400 mg, 1,500 mg, 1,600 mg, 1,700 mg, 1,800 mg, or 1,900 mg. In preferred embodiments, the composition comprises berberine hydrochloride in an amount of about 800 mg to 1,000 mg.

In embodiments, the composition comprises one or more omega-3 fatty acid, such as ALA, EPA, DHA, and/or combinations thereof in an amount of about 10 mg to 500 mg, such as about 20 mg, 30 mg, 40 mg, 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, or 475 mg. In preferred embodiments, the composition comprises DHA in an amount of about 200 mg to about 250 mg.

In embodiments, compositions can be administered as encapsulated products, tablets, or can be mixed into foods, shakes, flavored drink mixes, juices, yogurt, health bars, pastries, cereals, oatmeal, eggs, flavored water, or soups.

In embodiments, the composition is administered in dosages and at intervals capable of triggering hormones and metabolic processes capable of treating, preventing, and/or reversing metabolic syndrome, obesity, and/or one or more comorbidities thereof.

In embodiments, the composition is administered for a period of at least 2 weeks, such as 3 weeks, 4 weeks, 5 weeks, 6 weeks, 7 weeks, 8 weeks, 3 months, 6 months, 9 months, 1 year, or longer. In embodiments, preferable results are achieved after (or before) 4 weeks and even better results can be achieved after 8 weeks.

In embodiments, the composition is capable of reducing body weight by at least 0.5% after 4 weeks of administration, such as at least 1%, 2%, 3%, 4%, 5%, 8%, 10%, 15%, or more. In embodiments, the composition is capable of reducing body weight by up to about 30 pounds in 8 weeks, such as up to about 5 lbs., 8 lbs., 10 lbs., 12 lbs., 15 lbs., 18 lbs., 20 lbs., 22 lbs., 25 lbs., or 28 lbs.

In embodiments, the composition is capable of reducing LDL cholesterol and/or total cholesterol by up to about 20%, such as up to about 2%, 5%, 8%, 10, %, 12%, 15%, or 18%. In embodiments, the composition is capable of increasing HDL cholesterol by up to about 10%, such as up to about 2%, 3%, 4%, 5%, 6%, 7%, 8%, or 9%.

In embodiments, the composition is capable of reducing the risk of developing cardiovascular disease by up to about 20% after 8 weeks and/or by up to about 30% after about 16 weeks.

### EXAMPLES

The following Examples are illustrative and should not be interpreted to limit the scope of the claimed subject matter.

### Example 1

In an example study, subjects would be split into three groups. Group I would receive DHA (100 mg, administered twice daily), Group II would receive berberine hydrochloride (500 mg, administered twice daily), and Group III would receive a composition (administered twice daily) comprising both DHA (100 mg) and berberine hydrochloride (500 mg). Body weight would be measured at the onset of the trial, after 4 weeks, and after 8 weeks. The combination of DHA and Berberine shows unexpected synergy for the combination.

TABLE 1
Expected body weight reduction with administration of DHA, berberine,
and a composition comprising DHA and berberine. Weeks 4 and
8 show percent change in body weight from Week 0.

| | Week 4 | Week 8 |
|---|---|---|
| | (percent change | (percent change |
| | from Week 0) | from Week 0) |
| Group I - DHA (200 mg daily) | −0% | −0% |
| Group II - Berberine HCl (1000 | −2% | −4% |
| mg daily) | | |
| Group III - DHA (200 mg daily) + | −7% | −14% |
| Berberine HCl (1000 mg daily) | | |

TABLE 2
Expected cholesterol reduction with administration of DHA, berberine,
and a composition comprising DHA and berberine. Weeks 4 and
8 show percent change in cholesterol from week 0.

| | Week 4 | Week 8 |
|---|---|---|
| | (percent change | (percent change |
| | from Week 0) | from Week 0) |
| Group I - DHA (200 mg daily) | −0% | −1% |
| Group II - Berberine HCl (1000 | −2% | −4% |
| mg daily) | | |
| Group III - DHA (200 mg daily) + | −5% | −10% |
| Berberine HCl (1000 mg daily) | | |

### Example 2

In an embodiment, the composition comprises 1,000 mg of Berberine HCl and 200 mg of DHA. In embodiments, the composition may also comprise microcrystalline cellulose, Hypromellose (capsule), silica, and magnesium stearate. In aspects, a subject might take 4 capsules daily, such as two capsules with or around breakfast and two capsules at or around dinner.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary or explanatory in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1. A composition comprising:
a salt of berberine;
docosahexaenoic acid (DHA); and
optionally, one or more pharmaceutically acceptable excipients.

2. The composition of claim 1, wherein the salt of berberine is present in the composition in an amount in the range of about 800 mg to about 1,250 mg.

3. The composition of claim 1, wherein the DHA is present in the composition in an amount in the range of about 150 mg to about 350 mg.

4. The composition of claim 1, wherein the DHA is present in the composition in an amount of about 200 mg or about 250 mg, and wherein the salt of berberine is present in the composition in an amount of about 1,000 mg.

5. The composition of claim 1, wherein the salt of berberine is berberine chloride.

6. The composition of claim 1, wherein the composition is formulated as an oral dosage.

7. The composition of claim 6, wherein the oral dosage is a solid form or a liquid form.

8. The composition of claim 6, wherein the oral dosage is a pill, capsule, or tablet.

9. The composition of claim 1, wherein the salt of berberine, the docosahexaenoic acid (DHA), or both, are plant-based, mostly plant-based, or derived from one or more plants.

10. The composition of claim 1, wherein the composition is over about 95% by weight of the salt of berberine and the docosahexaenoic acid (DHA).

11. The composition of claim 1, wherein the salt of berberine and the docosahexaenoic acid (DHA) comprise over about 95% by weight of the composition.

12. The composition of claim 1, wherein the salt of berberine is a pharmaceutically acceptable salt.

13. A method, comprising:
administering to a subject in need thereof a composition comprising:
a salt of berberine; and
docosahexaenoic acid (DHA);
wherein the administering is performed to treat, prevent, reduce, or reverse one or more symptoms of one or more disease or condition.

14. The method of claim 13, wherein the salt of berberine is present in the composition in an amount in the range of about 800 mg to about 1,250 mg.

15. The method of claim 13, wherein the DHA is present in the composition in an amount in the range of about 150 mg to about 350 mg.

16. The method of claim 13, wherein the DHA is present in the composition in an amount of about 200 mg or about 250 mg, and wherein the salt of berberine is present in the composition in an amount of about 1,000 mg.

17. The method of claim 13, wherein the salt of berberine is berberine chloride.

18. The method of claim 13, wherein the composition is administered at least once per day, such as twice per day, three times per day, four times per day, or more.

19. The method of claim 13, wherein the salt of berberine and the DHA are present in the pharmaceutical composition in synergistically effective amounts.

20. The method of claim 13, wherein the composition is administered to the subject for a period of up to 8 weeks.

21. The method of claim 13, wherein the composition is administered to the subject for a period of at least 4 weeks.

22. The method of claim 13, wherein the one or more symptoms of one or more disease or condition, or the one or more disease or condition, is obesity.

23. The method of claim 13, wherein the one or more symptoms of one or more disease or condition, or the one or more disease or condition, is metabolic syndrome.

24. The method of claim 13, wherein the one or more symptoms of one or more disease or condition, or the one or more disease or condition, is cardiovascular disease.

25. The method of claim 13, wherein the one or more symptoms of one or more disease or condition, or the one or more disease or condition, is a slowed metabolism.

26. The method of claim 13, wherein the one or more symptoms of one or more disease or condition, or the one or more disease or condition, is type 2 diabetes.

27. The method of claim 13, wherein the one or more symptoms of one or more disease or condition is elevated blood sugar.

28. The method of claim 13, wherein, as a result of the administering, the subject experiences a decrease in body fat percentage, a decrease in body mass, and/or a reduction in waist circumference.

29. The method of claim 13, wherein as a result of the administering, the subject experiences an increase in lean muscle mass.

30. The method of claim 13, wherein as a result of the administering, the subject experiences an increased rate of wound healing.

31. The method of claim 13, wherein as a result of the administering, the subject experiences an increased metabolic rate.

32. The method of claim 13, wherein the salt of berberine is a pharmaceutically acceptable salt.
